(19) United States
(12) Patent Application Publication
(10) Pub. No.: US 2024/0164649 A1
(43) Pub. Date: May 23, 2024

(54) PHYSIOLOGICAL SIGNAL MEASURING METHOD AND SYSTEM THEREOF

(71) Applicant: NATIONAL YUNLIN UNIVERSITY OF SCIENCE AND TECHNOLOGY, YUNLIN COUNTY (TW)

(72) Inventors: Chuan-Yu CHANG, Yunlin County (TW); Yen-Qun GAO, Yunlin County (TW)

(21) Appl. No.: 18/324,999

(22) Filed: May 28, 2023

(30) Foreign Application Priority Data
Nov. 17, 2022 (TW) 111143990

(51) Int. Cl. A61B 5/01; G06V 40/16

(52) U.S. Cl. CPC A61B 5/01; G06V 40/171; G06V 2201/03

(57) ABSTRACT

A physiological signal measuring method includes a training's thermal image providing step, a training step, a classification model generating step, a measurement's thermal image providing step, a mask-wearing classifying step, a block identifying step and a measurement result generating step. The measurement's thermal image providing step includes providing a measurement's thermal image, which is an infrared thermal video for measuring. The measurement result generating step includes generating a measurement result of at least one physiological parameter of the subject according to a plurality of signals of the forehead block, and the mask block or the nasal cavity block.

### RELATED APPLICATIONS

This application claims priority to Taiwan Application Serial Number 111143990, filed Nov. 17, 2022, which is herein incorporated by reference.

### BACKGROUND

### Technical Field

The present disclosure relates to a physiological signal measuring method and a system thereof. More particularly, the present disclosure relates to a physiological signal measuring method and a system thereof applying an infrared thermal video.

### Description of Related Art

The most common way to measure the physiological parameter is to use a physiological monitor. However, contact measurement usually has concerns about contact infection, and the instruments need to be replaced or cleaned before each use.

The infrared thermography (IRT) is a non-contact measurement technology for the physiological parameter, and it is mainly used for measuring body temperature. During the COVID-19 epidemic, the technology is widely used to measure the body temperatures at the entrances and exits of the public places. However, if the infrared thermography can be used to measure more diverse physiological parameters, it will be advantageous in reducing the risks and inconveniences of contact measurement for the physiological parameters.

Given the above, how to effectively use infrared thermal images to measure more diverse physiological parameters has become an important topic of concern today.

### SUMMARY

According to one aspect of the present disclosure, a physiological signal measuring method includes a training's thermal image providing step, a training step, a classification model generating step, a measurement's thermal image providing step, a mask-wearing classifying step, a block identifying step and a measurement result generating step. The training's thermal image providing step includes providing a plurality of training's thermal images, which are a plurality of thermal images for training, each of the training's thermal images is an infrared thermal image, and each of the training's thermal images has a mark of a position of a person's face portion, and a mark of a mask-wearing state or a mark of a non-mask-wearing state. The training step includes training the training's thermal images by a machine learning algorithm. The classification model generating step includes generating a mask-wearing classification model after the training step by the machine learning algorithm, and a most accurate model weight obtained from the training step by the machine learning algorithm is used in the mask-wearing classification model. The measurement's thermal image providing step includes providing a measurement's thermal image, which is an infrared thermal video for measuring. The mask-wearing classifying step includes identifying a person's face portion of a subject in the measurement's thermal image by the mask-wearing classification model, and classifying the person's face portion as the mask-wearing state or the non-mask-wearing state. The block identifying step includes identifying a forehead block and a mask block in the person's face portion when the person's face portion is classified as the mask-wearing state, and identifying a forehead block and a nasal cavity block in the person's face portion when the person's face portion is classified as the non-mask-wearing state. The measurement result generating step includes generating a measurement result of at least one physiological parameter of the subject according to a plurality of signals of the forehead block, and the mask block or the nasal cavity block.

According to another aspect of the present disclosure, a physiological signal measuring system includes a thermographic unit, a processor and a storage medium. The thermographic unit is configured for providing a measurement's thermal image, which is an infrared thermal video for measuring. The processor is coupled to the thermographic unit. The storage medium is coupled to the processor and configured to provide a mask-wearing classification model and a physiological signal calculation program. Based on the mask-wearing classification model, the processor is configured to identify a person's face portion of a subject in the measurement's thermal image, and classify the person's face portion as a mask-wearing state or a non-mask-wearing state. Based on the physiological signal calculation program, the processor is configured to identify a forehead block and a mask block in the person's face portion when the person's face portion is classified as the mask-wearing state, identify a forehead block and a nasal cavity block in the person's face portion when the person's face portion is classified as the non-mask-wearing state, and generate a measurement result of at least one physiological parameter of the subject according to a plurality of signals of the forehead block, and the mask block or the nasal cavity block.

### BRIEF DESCRIPTION OF THE DRAWINGS

The present disclosure can be more fully understood by reading the following detailed description of the embodiment, with reference made to the accompanying drawings as follows:

FIG. 1A is a flow chart of a physiological signal measuring method according to the 1st embodiment of the present disclosure.

FIG. 1B is a partial flow chart of the physiological signal measuring method according to the 1st embodiment.

FIG. 1C is a schematic view of a forehead block of the 1st embodiment.

FIG. 1D is a schematic view of a mask block of the 1st embodiment.

FIG. 1E is a schematic view of a sliding window method in a ROI (region of interest) determining step of the 1st embodiment.

FIG. 1F is a schematic view of a forehead block and a nasal cavity block of the 1st embodiment.

FIG. 1G is a schematic view of a measurement result generating step of the 1st embodiment.

FIG. 2A is a block diagram of a physiological signal measuring system according to the 2nd embodiment of the present disclosure.

FIG. 2B is a schematic view of the physiological signal measuring system according to the 2nd embodiment.

### DETAILED DESCRIPTION

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein.

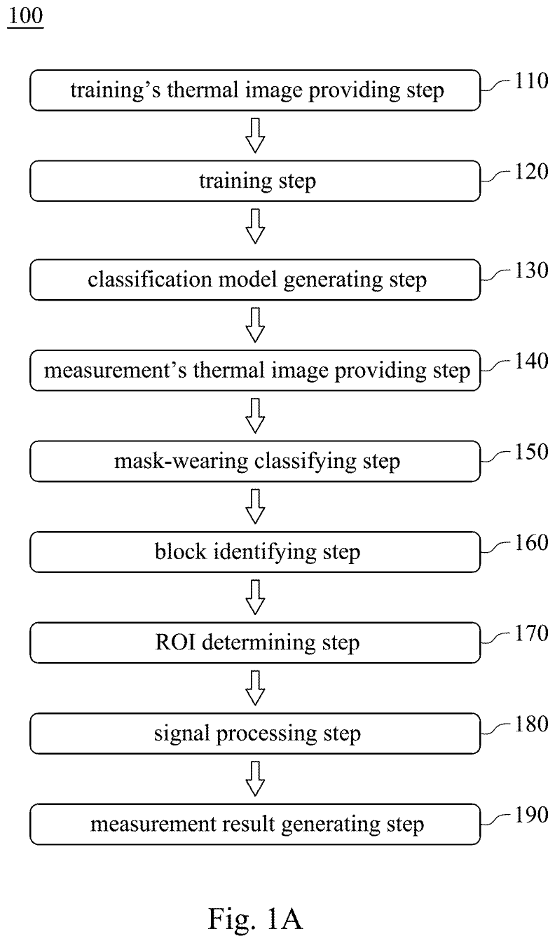

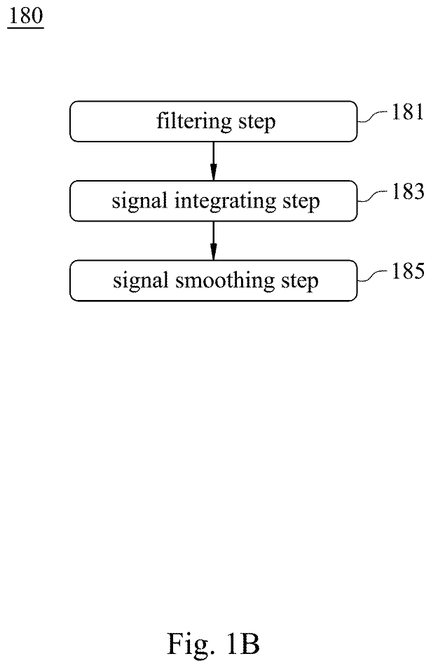

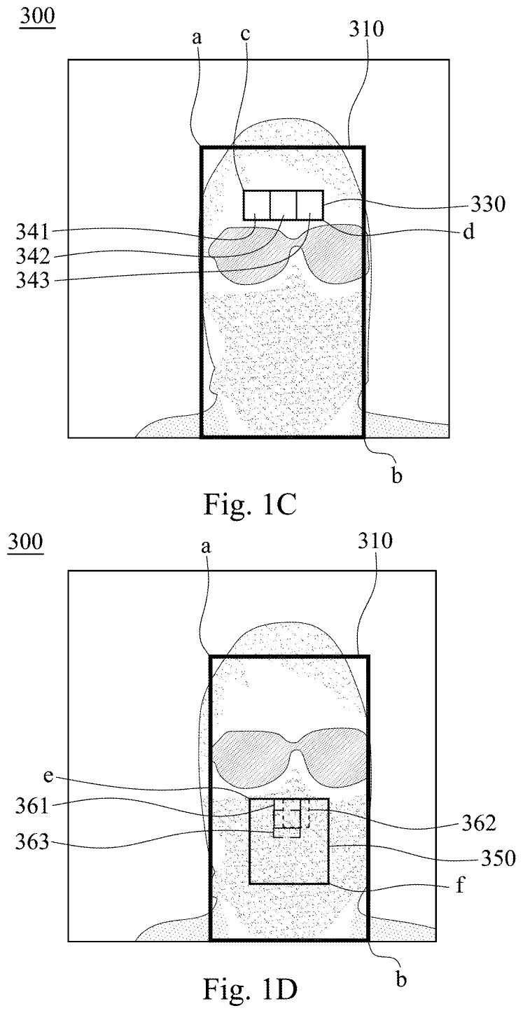

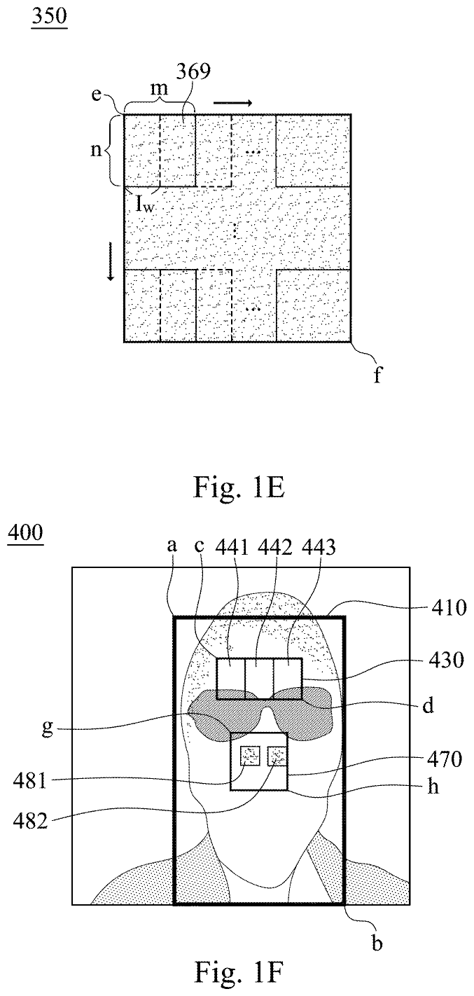

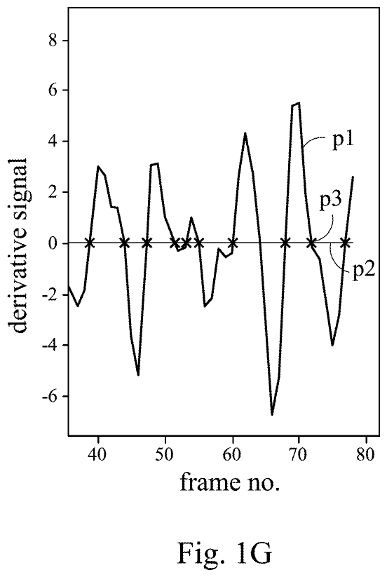

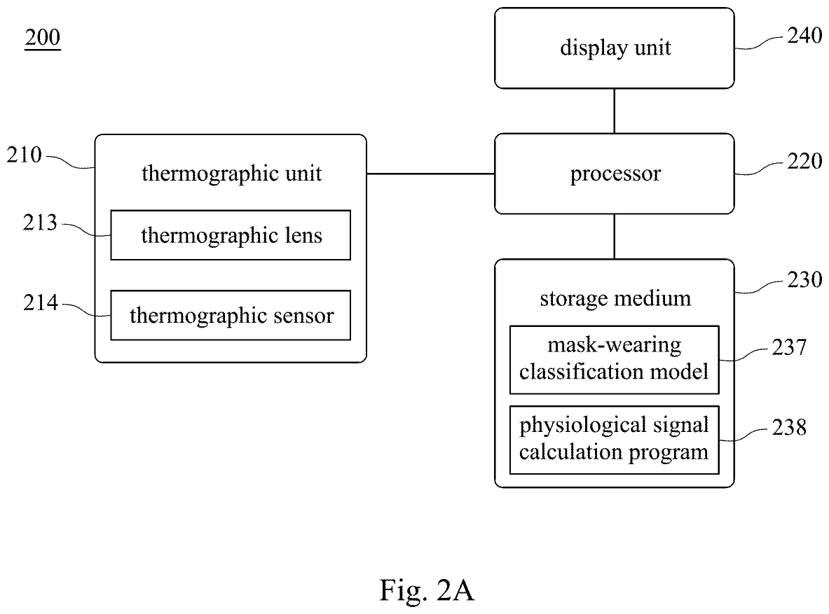

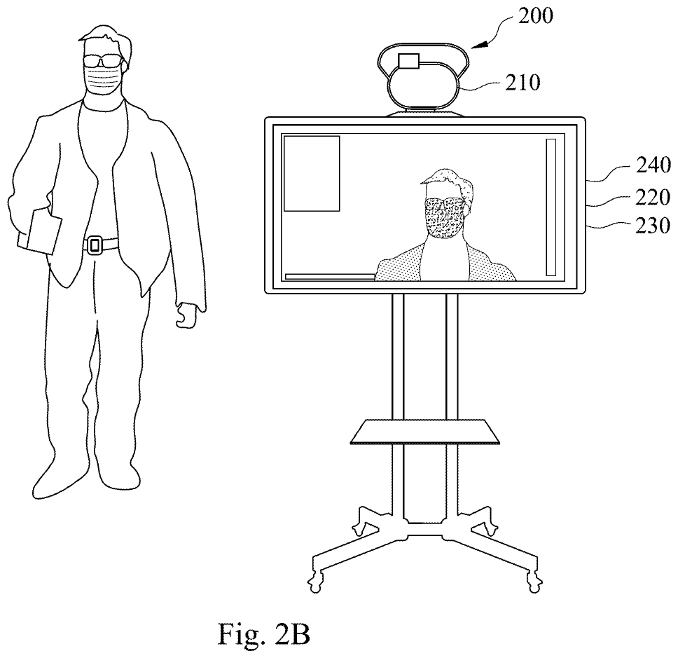

FIG. 1A is a flow chart of a physiological signal measuring method 100 according to the 1st embodiment of the present disclosure, FIG. 1B is a partial flow chart of the physiological signal measuring method 100 according to the 1st embodiment, FIG. 2A is a block diagram of a physiological signal measuring system 200 according to the 2nd embodiment of the present disclosure, and FIG. 2B is a schematic view of the physiological signal measuring system 200 according to the 2nd embodiment. With reference to FIGS. 1A, 1B, 2A and 2B, the physiological signal measuring system 200 according to the 2nd embodiment of the present disclosure is used to assist in describing the physiological signal measuring method 100 according to the 1st embodiment. The physiological signal measuring method 100 includes a training's thermal image providing step 110, a training step 120, a classification model generating step 130, a measurement's thermal image providing step 140, a mask-wearing classifying step 150, a block identifying step 160 and a measurement result generating step 190.

With reference to FIGS. 1A and 2A, the training's thermal image providing step 110 includes providing a plurality of training's thermal images, which are a plurality of thermal images for training, each of the training's thermal images is an infrared thermal image, and each of the training's thermal images has a mark of a position of a person's face portion, and a mark of a mask-wearing state or a mark of a non-mask-wearing state. The training step 120 includes training the training's thermal images by a machine learning algorithm. The classification model generating step 130 includes generating a mask-wearing classification model 237 after the training step 120 by the machine learning algorithm, and the mask-wearing classification model 237 may be a YOLOv5s model of a YOLO (You Only Look once) algorithm. A most accurate model weight obtained from the training step 120 by the machine learning algorithm is used in the mask-wearing classification model 237.

FIG. 1C is a schematic view of a forehead block 330 of the 1st embodiment, FIG. 1D is a schematic view of a mask block 350 of the 1st embodiment, FIG. 1E is a schematic view of a sliding window method in a ROI (region of interest) determining step 170 of the 1st embodiment, and FIG. 1F is a schematic view of a forehead block 430 and a nasal cavity block 470 of the 1st embodiment. With reference to FIGS. 1A, 1C to 1F and 2A, the measurement's thermal image providing step 140 includes providing, inputting, capturing or shooting measurement's thermal images 300, 400, and each of the measurement's thermal images 300, 400 is an infrared thermal video (i.e., film, continuous image frames) for measuring. The mask-wearing classifying step 150 includes identifying person's face portions 310, 410 of subjects in the measurement's thermal images 300, 400 by the mask-wearing classification model 237, and classifying each of the person's face portions 310, 410 as the mask-wearing state or the non-mask-wearing state.

The block identifying step 160 includes identifying a forehead block 330 and a mask block 350 in the person's face portion 310 as shown in FIGS. 1C and 1D when the person's face portion 310 is classified as the mask-wearing state, and identifying a forehead block 430 and a nasal cavity block 470 in the person's face portion 410 as shown in FIG. 1F when the person's face portion 410 is classified as the non-mask-wearing state. The measurement result generating step 190 includes generating a measurement result of at least one physiological parameter of the subject according to a plurality of signals of the forehead block 330 and the mask block 350 (or the forehead block 430 and the nasal cavity block 470). Therefore, the physiological signal measuring method 100 is advantageous in providing the accurate measurement results of the physiological parameters according to whether the mask is worn or not.

In the 1st embodiment, a number of the at least one physiological parameter may be at least three, and the physiological parameters include a body temperature, a heart rate and a respiration rate. The measurement result generating step 190 further includes generating a measurement result of the heart rate from a change of a forehead temperature (e.g., a period or a frequency corresponding to the change of the forehead temperature), and generating a measurement result of the respiration rate from a change of a mask temperature or a change of a nasal cavity temperature (e.g., a period or a frequency corresponding to the change of the mask temperature or the change of the nasal cavity temperature). Therefore, the physiological signal measurement method 100 is not only a non-contact measurement method for the physiological signals, which is not affected by ambient brightness, but also can be used for simultaneously measuring the body temperature, the heart rate, and the respiration rate.

In detail, in the mask-wearing state shown in FIGS. 1C and 1D, the physiological signal measuring method 100 may further include defining a coordinate system of the person's face portion 310 according to an upper left corner point a(0, 0) and a lower right corner point b(w, h), defining the forehead block 330 by two corner points c(w/4, h/7) and d(3w/4, 2h/7), and defining the mask block 350 by two corner points e(w/4, h/2) and f(3w/4, 4h/5). In the non-mask-wearing state shown in FIG. 1F, the physiological signal measuring method 100 may further include defining a coordinate system of the person's face portion 410 according to an upper left corner point a(0, 0) and a lower right corner point b(w, h), defining the forehead block 430 by two corner points c(w/4, h/7) and d(3w/4, 2h/7), and defining the nasal cavity block 470 by two corner points g(w/3, 2h/5) and h(2w/3, 3h/5). Therefore, the physiological signal measuring method 100 is beneficial to be applied to both the low-resolution thermographic devices and the high-resolution thermographic devices, and reduce product costs.

With reference to FIGS. 1A and 1C to 1F, the physiological signal measuring method 100 may further include the ROI determining step 170. The ROI determining step 170 include determining ROIs 341, 342, 343 of the forehead block 330 and ROIs 361, 362, 363 of the mask block 350, or include determining ROIs 441, 442, 443 of the forehead block 430 and ROIs 481, 482 of the nasal cavity block 470, and taking an average of a plurality of tracking signals of each of the ROIs 341, 342, 343, 361, 362, 363, 441, 442, 443, 481, 482 as an average tracking signal.

In the ROI determining step 170, specifically, the forehead block 330 may be evenly divided into three regions as the ROIs 341, 342, 343, and the forehead block 430 may be evenly divided into three regions as the ROIs 441, 442, 443, as shown in FIGS. 1C and 1F. In addition, as the mask block 350 shown in FIG. 1E for example, the signals of each of a plurality of windows 369 in the mask block 350 (or the nasal cavity block 470) may be tracked by a sliding window method. A width m and a height n of each of the sliding windows 369 may be ⅓ of a width and a height, respectively, of the mask block 350, and each of the windows 369 has a width step size lw and a height step size. Three (or two) of the windows 369 that have maximum changes after a variance calculating are defined as the ROIs 361, 362, 363 of the mask block 350 (or the ROIs 481, 482 of the nasal cavity block 470). Thus, the measurement result generating step 190 may further include generating the measurement result of the at least one physiological parameter of the subject according to the average tracking signals of the ROIs 341, 342, 343, 361, 362, 363, respectively, of the forehead block 330 and the mask block 350 (or according to the average tracking signals of the ROIs 441, 442, 443, 481, 482, respectively, of the forehead block 430 and the nasal cavity block 470). Therefore, it is advantageous in obtaining the heart rate and the respiration rate from subsequently processing the temperature changes of the forehead blocks 330, 430 and the temperature changes of the mask block 350 and the nasal cavity block 470, respectively.

Furthermore, a number of the ROIs of the mask block 350 may be three, that is, the ROIs 361, 362, 363. A number of the ROIs of the nasal cavity block 470 may be two, that is, the ROIs 481, 482, which match a left nostril and a right nostril, respectively. Therefore, an appropriate number of the ROIs is beneficial to efficiently achieve the accurate measurements.

With reference to FIGS. 1A and 1B, the physiological signal measuring method 100 may further include a signal processing step 180, which includes a filtering step 181, a signal integrating step 183 and a signal smoothing step 185.

With reference to FIGS. 1B to 1F, the filtering step 181 includes processing each of the average tracking signals by at least one bandpass filtering algorithm, e.g., Butterworth filter (BF), and generating a filtered signal. Specifically, the average tracking signal of each of the ROIs 341, 342, 343 of the forehead block 330 (or each of the ROIs 441, 442, 443 of the forehead block 430) is processed by a bandpass filtering algorithm with a pass band of 0.75 Hz to 3.0 Hz (corresponding to 45 to 180 times per minute) to generate a filtered signal, and the average tracking signal of each of the ROIs 361, 362, 363 of the mask block 350 (or each of the ROIs 481, 482 of the nasal cavity block 470) is processed by a bandpass filtering algorithm with a pass band of 0.15 Hz to 0.5 Hz (corresponding to 9 to 30 times per minute) to generate a filtered signal. Therefore, it is advantageous in obtaining the heart rate and the respiration rate from subsequently processing the temperature changes of the forehead blocks 330, 430 and the temperature changes of the mask block 350 and the nasal cavity block 470, respectively.

The signal integrating step 183 includes integrating the filtered signals of the ROIs 341, 342, 343 of the forehead block 330 (or the ROIs 441, 442, 443 of the forehead block 430) into a principal signal, and integrating the filtered signals of the ROIs 361, 362, 363 of the mask block 350 (or the ROIs 481, 482 of the nasal cavity block 470) into another principal signal. Each of the principal signals is the most principal component calculated by the principal component analysis (PCA). The signal smoothing step 185 includes smoothing each of the principal signals by morphological filtering and generating a smoothed signal. Therefore, the smoothed signals do not cause to reduce the peak energy due to filtering, which will result in measurement errors.

FIG. 1G is a schematic view of the measurement result generating step 190 of the 1st embodiment. With reference to FIGS. 1A to 1G, the measurement result generating step 190 may further include generating the measurement results of the physiological parameters of the subject according to the smoothed signals. In detail, each of the smoothed signals is converted to a derivative signal p1 by a first derivative method, a plurality of time intervals formed by a plurality of intersection points p3 intersected by each of the derivative signals p1 (may be normalized derivative signals) and a zero-cross line p2 are calculated and obtained, an average time interval among the intersection points p3 is calculated and obtained and then converted into the heart rate per minute and the respiration rate per minute, which are the measurement results of the physiological parameters of the subject. The heart rate is generated by processing the signals of the forehead block 330 or 430, and the respiration rate is generated by processing the signals of the mask block 350 or the nasal cavity block 470. Therefore, it can avoid the problem of selecting a noise resulting in the surge while it is too difficult to determine the peak in the peak detection method. Furthermore, the first derivative method is advantageous in signal troughs to appear normal zero-cross conditions and simultaneously suppressing the peaks caused by the noises.

With reference to FIGS. 1A, 1C to 1F, 2A and 2B, the physiological signal measuring system 200 according to the 2nd embodiment of the present disclosure includes a thermographic unit 210, a processor 220 and a storage medium 230. The thermographic unit 210 including a thermographic lens 213 and a thermographic sensor 214 is configured for providing the measurement's thermal images 300, 400, which are the infrared thermal videos for measuring. The processor 220 is coupled to the thermographic unit 210. The storage medium 230 is coupled to the processor 220 and configured to provide the mask-wearing classification model 237 and a physiological signal calculation program 238. Furthermore, the physiological signal measuring system 200 may further include a display unit 240, which is coupled to the processor 220 and configured to display the measurement's thermal images 300, 400. The storage medium 230 may specifically be a local or cloud non-transitory computer-readable storage medium, and the thermographic unit 210, the processor 220, the storage medium 230 and the display unit 240 may specifically be in a form of multiple devices or integrated into a single device.

Based on the mask-wearing classification model 237, the processor 220 is configured to identify the person's face portions 310, 410 of the subjects in the measurement's thermal images 300, 400, and classify each of the person's face portions 310, 410 as the mask-wearing state or the non-mask-wearing state.

Based on the physiological signal calculation program 238, the processor 220 is configured to identify the forehead block 330 and the mask block 350 in the person's face portion 310 as shown in FIGS. 1C and 1D when the person's face portion 310 is classified as the mask-wearing state, identify the forehead block 430 and the nasal cavity block 470 in the person's face portion 410 as shown in FIG. 1F when the person's face portion 410 is classified as the non-mask-wearing state, and generate the measurement results of the physiological parameters of the subjects according to the signals of the forehead block 330 and the mask block 350 (or the forehead block 430 and the nasal cavity block 470). Therefore, the physiological signal measuring system 200 is advantageous in providing the accurate measurement results of the physiological parameters according to whether the mask is worn or not.

In detail, the mask-wearing classification model 237 may be generated from training the training's thermal images by the machine learning algorithm, and the most accurate model weight obtained from training by the machine learning algorithm is used in the mask-wearing classification model 237.

Regarding other details of the physiological signal measuring system 200 of the 2nd embodiment, the contents of the physiological signal measuring method 100 of the 1st embodiment can be referred, and the details are not described again herein.

Although the present disclosure has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A physiological signal measuring method, comprising:
a training's thermal image providing step comprising providing a plurality of training's thermal images, which are a plurality of thermal images for training, wherein each of the training's thermal images is an infrared thermal image, and each of the training's thermal images has a mark of a position of a person's face portion, and a mark of a mask-wearing state or a mark of a non-mask-wearing state;
a training step comprising training the training's thermal images by a machine learning algorithm;
a classification model generating step comprising generating a mask-wearing classification model after the training step by the machine learning algorithm, wherein a most accurate model weight obtained from the training step by the machine learning algorithm is used in the mask-wearing classification model;
a measurement's thermal image providing step comprising providing a measurement's thermal image, which is an infrared thermal video for measuring;
a mask-wearing classifying step comprising identifying a person's face portion of a subject in the measurement's thermal image by the mask-wearing classification model, and classifying the person's face portion as the mask-wearing state or the non-mask-wearing state;
a block identifying step comprising identifying a forehead block and a mask block in the person's face portion when the person's face portion is classified as the mask-wearing state, and identifying a forehead block and a nasal cavity block in the person's face portion when the person's face portion is classified as the non-mask-wearing state; and
a measurement result generating step comprising generating a measurement result of at least one physiological parameter of the subject according to a plurality of signals of the forehead block, and the mask block or the nasal cavity block.

2. The physiological signal measuring method of claim 1, further comprising:
a ROI (region of interest) determining step comprising determining a plurality of ROIs of each of the forehead block, and the mask block or the nasal cavity block, and taking an average of a plurality of tracking signals of each of the ROIs as an average tracking signal, wherein the signals of each of a plurality of windows in the mask block or the nasal cavity block are tracked by a sliding window method, and ones of the windows that have maximum changes after a variance calculating are defined as the ROIs of the mask block or the nasal cavity block;
wherein the measurement result generating step further comprising generating the measurement result of the at least one physiological parameter of the subject according to the average tracking signals of the ROIs, respectively, of the forehead block, and the mask block or the nasal cavity block.

3. The physiological signal measuring method of claim 2, further comprising:
a signal processing step comprising:
a filtering step comprising processing each of the average tracking signals by at least one bandpass filtering algorithm and generating a filtered signal;
a signal integrating step comprising integrating the filtered signals of the ROIs, respectively, of each of the forehead block, and the mask block or the nasal cavity block into a principal signal; and
a signal smoothing step comprising smoothing each of the principal signals and generating a smoothed signal;
wherein the measurement result generating step further comprising generating the measurement result of the at least one physiological parameter of the subject according to the smoothed signals.

4. The physiological signal measuring method of claim 1, wherein a number of the at least one physiological parameter is at least three, and the physiological parameters comprise a body temperature, a heart rate and a respiration rate;
wherein the measurement result generating step further comprising generating a measurement result of the heart rate from a change of a forehead temperature, and generating a measurement result of the respiration rate from a change of a mask temperature or a change of a nasal cavity temperature.

5. A physiological signal measuring system, comprising:
a thermographic unit configured for providing a measurement's thermal image, which is an infrared thermal video for measuring;
a processor coupled to the thermographic unit; and
a storage medium coupled to the processor and configured to provide a mask-wearing classification model and a physiological signal calculation program;
wherein based on the mask-wearing classification model, the processor is configured to:
identify a person's face portion of a subject in the measurement's thermal image, and classify the person's face portion as a mask-wearing state or a non-mask-wearing state;
wherein based on the physiological signal calculation program, the processor is configured to:
identify a forehead block and a mask block in the person's face portion when the person's face portion is classified as the mask-wearing state, and identify a forehead block and a nasal cavity block in the person's face portion when the person's face portion is classified as the non-mask-wearing state; and
generate a measurement result of at least one physiological parameter of the subject according to a plurality of signals of the forehead block, and the mask block or the nasal cavity block.

6. The physiological signal measuring system of claim 5, wherein the mask-wearing classification model is generated from training a plurality of training's thermal images, which are a plurality of thermal images for training, by a machine learning algorithm, and a most accurate model weight obtained from training by the machine learning algorithm is used in the mask-wearing classification model.

7. The physiological signal measuring system of claim 5, wherein based on the physiological signal calculation program, the processor is further configured to:
define a coordinate system of the person's face portion according to an upper left corner point (0, 0) and a lower right corner point (w, h), define the forehead block by two corner points (w/4, h/7) and (3w/4, 2h/7), define the mask block by two corner points (w/4, h/2) and (3w/4, 4h/5), and define the nasal cavity block by two corner points (w/3, 2h/5) and (2w/3, 3h/5);
determine a plurality of ROIs of each of the forehead block, and the mask block or the nasal cavity block, and take an average of a plurality of tracking signals of each of the ROIs as an average tracking signal, wherein the signals of each of a plurality of windows in the mask block or the nasal cavity block are tracked by a sliding window method, and ones of the windows that have maximum changes after a variance calculating are defined as the ROIs of the mask block or the nasal cavity block; and
generate the measurement result of the at least one physiological parameter of the subject according to the average tracking signals of the ROIs, respectively, of the forehead block, and the mask block or the nasal cavity block.

8. The physiological signal measuring system of claim 7, wherein a number of the at least one physiological parameter is at least three, a number of the ROIs of the nasal cavity block is two, and the two ROIs of the nasal cavity block match a left nostril and a right nostril, respectively.

9. The physiological signal measuring system of claim 7, wherein based on the physiological signal calculation program, the processor is further configured to:
process the average tracking signal of each of the ROIs of the forehead block by a bandpass filtering algorithm with a pass band of 0.75 Hz to 3.0 Hz and generate a filtered signal, and process the average tracking signal of each of the ROIs of the mask block or the nasal cavity block by a bandpass filtering algorithm with a pass band of 0.15 Hz to 0.5 Hz and generate a filtered signal;
integrate the filtered signals of the ROIs, respectively, of each of the forehead block, and the mask block or the nasal cavity block into a principal signal;
smooth each of the principal signals by morphological filtering and generate a smoothed signal; and
convert each of the smoothed signals to a derivative signal by a first derivative method, calculate a plurality of time intervals formed by a plurality of intersection points intersected by each of the derivative signals and a zero-cross line, and generate the measurement result of the at least one physiological parameter of the subject.

10. The physiological signal measuring system of claim 5, wherein a number of the at least one physiological parameter is at least three, and the physiological parameters comprise a body temperature, a heart rate and a respiration rate;
wherein based on the physiological signal calculation program, the processor is further configured to:
generate a measurement result of the heart rate from a change of a forehead temperature, and generate a measurement result of the respiration rate from a change of a mask temperature or a change of a nasal cavity temperature.
